(19) United States
(12) Patent Application Publication
(10) Pub. No.: US 2026/0315053 A1
(43) Pub. Date: Oct. 15, 2026

(54) DYNAMIC TISSUE TRACTION DEVICES, SYSTEMS, AND METHODS

(71) Applicants: Boston Scientific Scimed, Inc., Maple Grove, MN (US); Boston Scientific Medical Device Limited, Galway (IE)

(72) Inventors: Julie Gagnon, Providence, RI (US); Deepak Kumar Sharma, Muzzaffarnagar (IN); Juan Pablo Ortiz Garcia, Heredia (CR); Colby Harris, Norfolk, MA (US)

(73) Assignees: Boston Scientific Scimed, Inc., Maple Grove, MN (US); Boston Scientific Medical Device Limited, Galway (IE)

(21) Appl. No.: 19/641,871

(22) Filed: Apr. 8, 2026

Related U.S. Application Data
(60) Provisional application No. 63/785,897, filed on Apr. 9, 2025.

(51) Int. Cl. A61B 17/00; A61B 1/00

(52) U.S. Cl. CPC A61B 17/00234; A61B 1/00119; A61B 1/0014; A61B 2017/00269; A61B 2017/00296; A61B 2017/00477

(57) ABSTRACT

A tissue traction system and associated devices and method for applying traction to target tissue within a patient’s body by applying a force to an elongate element operably associated with the target tissue. The system may include two or more tissue-engagement elements operably associated with the elongate element. One of the tissue-engagement elements may be engaged with the target tissue, and another tissue-engagement element may be engaged with tissue at a vector-changing location spaced apart from the target tissue. The elongate element is fixedly coupled to the anchor tissue-engagement element to apply traction to the target tissue via the anchor tissue-engagement element, and is movably operably associated with a traction-adjusting tissue-engagement element to modify the traction force vector applied to the target tissue. The system may be delivered external to a working channel of a medical delivery device such as a medical scope.

### CROSS-REFERENCE TO RELATED APPLICATIONS

This application claims the benefit of priority under 35 U.S.C. § 119 to U.S. Provisional Application No. 63/785,897, filed on Apr. 9, 2025, the entire disclosure of which is hereby incorporated by reference herein for all purposes.

### FIELD

The present disclosure relates generally to the field of medical devices, systems, and methods for applying traction to tissue.

### BACKGROUND

Endoscopic Mucosal Resection (EMR) is an endoscopic procedure which involves the placement of a snare around tissue and either contracting the snare to transect (cold), or using electrosurgical current to transect (hot) the encircled tissue. Endoscopic Submucosal Dissection (ESD) is an endoscopic procedure, first developed in Japan in the late 1990’s, used to remove superficial gastrointestinal neoplasms via en bloc resection as a reaction to the shortcomings of EMR. Typically, ESD procedures are technically more challenging than EMR procedures, with longer procedural times and higher rates of complications. However, en bloc resection allows good histopathological assessment of lesion margins, lower recurrence rates, and a greater minimally invasive potential for cure.

Minimally invasive (e.g., transluminal, transcatheter, endoscopic, etc.) surgical techniques like ESD and EMR typically allow for faster recovery than with open or laparoscopic surgical procedures. However, because such procedures are minimally invasive, there is limited space to maneuver within the body, and such procedures typically require a high degree of expertise. In procedures involving cutting of tissue, one of the largest time and complexity drivers is managing the tissue being cut. The loose section of tissue may obstruct visibility, such as by falling on the instruments being used, occluding visibility of the imaging device (e.g., camera), and creating a hindrance affecting movement of the instruments used during the procedure and in reaching all regions and depths of the target tissue being cut. Various solutions for lifting the cut (and often hanging) mass of tissue, thus clearing the path for visibility and operation of medical tools and devices, have been developed. However, positioning of the elements used with such solutions may be challenging, particularly in a space-restricted environment. Moreover, as tissue is cut, a continual increase in traction is required, presenting various challenges during the procedure. Additionally, the devices used with such solutions may require separate medical tools than those used to perform the procedure, and such tools may even require a separate working channel, thereby potentially increasing the size and/or complexity of the delivery system. As endoscopic submucosal dissection continues to evolve with new techniques, and new instruments are designed for making the procedure faster and safer, there remains a need for improvements to application of traction to tissue during the ESD procedure. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In some aspects, a tissue traction system formed in accordance with various principles of the present disclosure includes two or more tissue-engagement elements each configured to engage tissue within a patient’s body; and at least one elongate element. In some aspects, the two or more tissue-engagement elements include at least one anchor tissue-engagement element configured to engage target tissue within a patient’s body to which traction is to be applied, and at least one traction-adjusting tissue-engagement element; the at least one elongate element is fixedly coupled to the anchor tissue-engagement element; at least the anchor tissue-engagement element is deployed from a delivery device to be separated from the delivery device when engaged with the target tissue; and the elongate element is movably operably associated with the at least one traction-adjusting tissue-engagement element to adjust a traction force vector applied to the anchor tissue-engagement element by the elongate element to apply traction to the target tissue.

In some aspects, the tissue traction system further includes a tubular elongate member defining a lumen therethrough, wherein the two or more tissue-engagement elements are deliverable within the lumen of the tubular elongate member to a target site within the patient’s body. In some aspects, the tubular elongate member is configured to be operably associated with a medical delivery device configured to deliver a medical instrument to the target site. In some aspects, the tubular elongate member is operably associated with an exterior of the medical delivery device for delivery alongside the exterior of the medical delivery device with the lumen of the tubular elongate member providing an auxiliary lumen to the medical delivery device. In some aspects, the tissue traction system further includes a push rod longitudinally translatable within the lumen of the tubular elongate member to distally advance the tissue-engagement elements out of the tubular elongate member.

In some aspects, the at least one traction-adjusting tissue-engagement element includes at least a first traction-adjusting tissue-engagement element and a second traction-adjusting tissue-engagement element, and the at least one elongate element includes a first elongate element movably coupled with the first traction-adjusting tissue-engagement element and a second elongate element movably coupled with the second traction-adjusting tissue-engagement element. In some aspects, the first elongate element and the second elongate element are both fixedly coupled with the at least one anchor tissue-engagement element. In some aspects, the at least one anchor tissue-engagement element, the first traction-adjusting tissue-engagement element, and the second traction-adjusting tissue-engagement element are deliverable in series within a lumen defined in a tubular elongate member. In some aspects, the at least one anchor tissue-engagement element comprises a first anchor tissue-engagement element and a second anchor tissue-engagement element; the first elongate element is fixedly coupled with the first anchor tissue-engagement element; and the second elongate element is fixedly coupled with the second anchor tissue-engagement element. In some aspects, the tissue traction system further includes a first tubular elongate member configured for delivering the first anchor tissue-engagement element, and a second tubular elongate member configured for delivering the first traction-adjusting tissue-engagement element the second anchor tissue-engagement element, and the second anchor tissue-engagement element. In some aspects, the plurality of tubular elongate members are operably associable about the circumference of a medical delivery device.

In some aspects, the at least one elongate element is fixedly coupled with respect to an anchor point along the anchor tissue-engagement element.

In some aspects, the at least one elongate element is slidably associated with the at least one traction-adjusting tissue-engagement element. In some aspects, the at least one elongate element is fixedly coupled to an anchor point on the anchor tissue-engagement element to transmit a traction force to the anchor tissue-engagement element to apply traction to the target tissue, and is slidable through a translation point configured to capture the elongate element with respect to the traction-adjusting tissue-engagement element yet allow translation of the elongate element with respect to the translation point.

In some aspects, a tissue traction system formed in accordance with various principles of the present disclosure includes two or more tissue-engagement elements each configured to engage tissue within a patient’s body; at least one elongate element; and a tubular elongate member configured to deliver the two or more tissue-engagement elements and the at least one elongate element into a patient’s body. In some aspects, the two or more tissue-engagement elements include at least one anchor tissue-engagement element configured to engage target tissue within a patient’s body to which traction is to be applied, and at least one traction-adjusting tissue-engagement element; the at least one elongate element is fixedly coupled to the anchor tissue-engagement element; at least the anchor tissue-engagement element is deployed from a delivery device to be separated from the delivery device when engaged with the target tissue; and the elongate element is movably operably associated with the at least one traction-adjusting tissue-engagement element to adjust a traction force vector applied to the anchor tissue-engagement element by the elongate element to apply traction to the target tissue.

In some aspects, the tubular elongate member is flexible to be navigated within curved passages within the patient’s body, and defines a lumen therethrough, wherein the two or more tissue-engagement elements are deliverable in series within the lumen of the tubular elongate member to a target site within the patient’s body.

In some aspects, the tissue traction system further includes a plurality of tubular elongate members operably associable with a medical delivery device, in which the at least one anchor tissue-engagement element comprises a first anchor tissue-engagement element deliverable by a first of the plurality of tubular elongate members; and the at least one traction-adjusting tissue-engagement element comprises a first traction-adjusting tissue-engagement element deliverable by a second of the plurality of tubular elongate members. In some aspects, the tissue traction system further includes a second anchor tissue-engagement element deliverable by a third of the plurality of tubular elongate members; and a second traction-adjusting tissue-engagement element deliverable by a fourth of the plurality of tubular elongate members.

In some aspects, a method of applying traction to target tissue within a patient’s body in accordance with various principles of the present disclosure includes deploying an anchor tissue-engagement element at a target site within the patient’s body by separating the anchor tissue-engagement element from a first delivery device and engaging the anchor tissue-engagement element with target tissue at the target site; deploying a traction-adjusting tissue-engagement element by separating the traction-adjusting tissue-engagement element from a delivery device, which is the same as or delivered with the first delivery device, and engaging the traction-adjusting tissue-engagement element with tissue at a vector-changing location within the patient’s body spaced apart from the target site; and extending an elongate element, having a distal end fixedly coupled to the anchor tissue-engagement element, from the anchor tissue-engagement element to the traction-adjusting tissue-engagement element; and moving the elongate element with respect to the traction-adjusting tissue-engagement element to adjust a traction force vector applied by the elongate element to the anchor tissue-engagement element to apply traction to the target tissue. In some aspects, the anchor tissue-engagement element and the traction-adjusting tissue-engagement element are delivered external to a medical delivery device defining a lumen through which additional medical instruments are deliverable to the target site, and the elongate element extends external to the medical delivery device, the method comprising applying a traction force to the elongate element from outside the patient’s body.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable but is intended to be scaled down in relation to a device sized and proportioned to fit within a patient’s body, such as by a transluminal, minimally invasive delivery. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a tissue traction system formed in accordance with aspects of the present disclosure.

FIGS. 2A-2D illustrate delivery and deployment and use of a tissue traction system such as illustrated in FIG. 1, within a schematic illustration of a target site within a patient.

FIG. 3A is a cross-sectional view along a tubular elongate member delivering tissue-engagement elements formed in accordance with various principles of the present disclosure.

FIG. 3B is a view similar to the view of FIG. 3A but with a distalmost tissue-engagement element being shifted into a tissue-receiving configuration.

FIG. 3C is a detail view of FIG. 3B, at a different perspective angle, illustrating actuation of the tissue-engagement element to shift into the tissue-receiving configuration.

FIG. 4 illustrates an example of an embodiment of a tissue traction system formed in accordance with various principles of the present disclosure and deployed in a schematic illustration of a target site within a patient.

FIG. 5 illustrates a perspective view of an example of an embodiment of a tissue traction system formed in accordance with various principles of the present disclosure.

FIG. 6 illustrates delivery devices with which a tissue traction system such as illustrated in FIG. 5 may be delivered.

FIG. 7 illustrates a perspective view of an example of an embodiment of a tissue traction system similar to the tissue traction system illustrated in FIG. 5, and illustrating, in phantom, components of a tissue-engagement element not visible in FIG. 5.

FIGS. 8A-8C illustrate delivery and deployment and use of a tissue traction system such as illustrated in FIGS. 5-7, within a schematic illustration of a target site within a patient.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

There has been a growing interest in the medical field in minimally-invasive procedures, e.g., transluminal, transcatheter, endoscopic, etc., which do not require open surgery (cutting open the patient), but, instead, access a target site within the patient via a natural orifice (or, in some instances, a small incision not considered to constitute an open-surgery cut). Various endoluminal surgical (ELS) procedures have been developed, such as, without limitation, Endoscopic Mucosal Resection (EMR), Endoscopic Submucosal Dissection (ESD), to be performed within a patient and not in direct view of the medical professionals performing the procedure. The lack of direct visibility, as well as typical limited space for maneuvering of medical instruments presents various challenges to the medical professionals performing the procedure.

Although EMR can be performed in piecemeal technique, pathology on tissue fragments is often challenging and patients require more frequent follow-up endoscopies due to higher rate of incomplete resections and recurrences. While EMR is associated with relatively low technical complexity, shorter procedure times, and a low risk of adverse events, en bloc EMR is limited typically to lesions which are less than 2.0cm in diameter/width. More specifically, for large sessile or laterally spreading polyps, en bloc resection with convention EMR is nearly impossible for lesions greater than 2cm. For colorectal lesions of greater than 2cm, en bloc resection can only be achieved in about 30% of cases.

Medical professionals thus often turn to ESD for resection of challenging lesions, as well as large lesions. It is not uncommon for ESD to be utilized to remove neoplasms between 2 - 7cm. The European Society of Gastrointestinal Endoscopy (ESGE) recommends ESD be used first line in the removal of Gastric Superficial neoplastic lesions with very low risk of lymph node metastases (EMR may be considered if the lesion is less than 10 to 15mm with a very low probability of advanced histology). Often, ESD is also recommended first line for superficial esophageal squamous cell cancer (with EMR an option for lesions which are less than 10 mm). For colonic and rectal lesions, ESGE recommends removal of lesions with polypectomy or EMR. However, ESD can be considered if there is a high suspicion of limited submucosal invasion (indicated by depressed morphology and an irregular or non-granular surface pattern), and especially if the lesion is greater than 20mm, or in lesions that otherwise cannot be optimally and radically removed by snare-based techniques (such as EMR techniques).

Although ESD has been in practice for two decades, ESD remains a very technically challenging procedure. In Japan, where ESD procedure is best established, the complication rates (including perforation, peritonitis, and bleeding) are 3.5% for gastric ESD, 3.3% for esophageal ESD, and 4.6% for Colorectal ESD. Outside of Japan and China, EDS is practiced by a relatively small number of highly technically skilled endoscopists and surgeons. Long procedure times and high risk of perforation are still significant challenges which have prevented broad physician adoption.

In accordance with various principles of the present disclosure, devices, systems, and methods are described which may be used in minimally invasive procedures, such as ESD, to facilitate manipulation of an anatomical structure within the patient’s body. The anatomical structure may be anatomical tissue, and the device may be configured to move a portion or layer of tissue relative to another portion or layer of tissue. It will be appreciated that reference may be made herein simply to “tissue” instead of “anatomical tissue” or “anatomical structure” for the sake of convenience and without intent to limit. In some aspects, the present disclosure is directed to devices, systems, and methods for moving, such as to separate and/or lift and/or apply traction to tissue within a patient’s body.

Traction is a foundational technique utilized in open and laparoscopic surgery. Traction may be used to move tissue or other anatomical structures and may be advantageously used to greatly enhance visualization of anatomy, tissue planes, and potential hazards such as nerves or blood vessels. Additionally or alternatively, traction may be used to impart a tension force to the tissue, which may change the way the tissue behaves as it is being cut or dissected. For instance, traction may be used in surgical procedures to “present” the tissue to be cut to the cutting instrument in an orientation or manner that makes the cutting easier and/or helps to ensure that the cut is made through the desired tissue in the desired direction. In some aspects, traction alters the nature and/or properties of tissue in a manner which may facilitate performance of a procedure with respect to or in the vicinity of the tissue under traction. It will be appreciated that principles of the present disclosure are applicable to tissue which is cut as well as tissue or other anatomical structures which are not cut. For instance, principles of the present disclosure are applicable for providing traction and/or counter-traction during various medical procedures, including, without limitation, surgical procedures that involve cutting of tissue. The use of traction is a basic precept of surgery during cutting procedures, but should be appreciated as having broader applicability as well.

In addition to the procedural benefits of using traction during endoscopic procedures, there is a mounting body of evidence that devices aiding with traction during endoscopic submucosal dissection reduce the technical complexity of the procedure, and significantly lower the learning curve associated with trainees and/or new ESD procedure adopters.

To date, endoscopic traction solutions, both commercialized and physician made, do not adequately address true user needs. At best, commercially available traction solutions leave significant room for improvement with regard to efficiency, effectiveness, flexibility, and ease of use. In open surgery, traction typically is applied using the non-dominant hand directly or through a surgical instrument (typically some kind of forceps). In laparoscopic surgery, traction is applied via tissue graspers introduced through one of the surgical ports, which provide access to the target body cavity or space. This can be done by the lead surgeon, assisting surgeon, or scrub nurse. In endoscopic surgery, however, multiple portals for accessing the target surgical site generally are not provide, and, typically, only the endoscope (or other access device) provides both visualization and access. Although there are some endoscopes with two working channels, or devices that can attach to the outside of the endoscope to provide a secondary working channel, all of these access channels are either integral to the endoscope or run parallel to it, and therefore are directionally constrained, and limited in the degrees of freedom of movement and control that can be achieved when maneuvering other devices to the target site.

The present disclosure offers various advantages over current traction devices and techniques. In some aspects, principles of the present disclosure allow for adjustability of a traction force applied to target tissue at a target site within a patient’s body via an elongate element. In some aspects, the elongate member extends to a proximal end positioned outside the patient’s body or is operably associated with a member positioned outside the patient’s body so that a medical professional may apply traction force to the elongate element for application and/or modification of a traction force to target tissue with which the elongate element is operably associated. It will be appreciated that the elongate element may be any elongate element suitable for transmitting traction to tissue (directly or via another element), such as, without limitation, a tether, filament, string, cord, wire, band, loop, dental floss, etc., either elastic or inelastic, stretchable or not stretchable, metallic or polymeric or combination thereof, etc., and typically sufficiently flexible to be navigated through curved passages (e.g., within the patient). It will be appreciated that reference may be made to a treatment site, anatomical site, delivery site, deployment site, implant / implantation site, site of implantation, target site, etc., interchangeably and without intent to limit. In some aspects, target tissue is defined within the treatment site and with respect to which a procedure is to be performed. The target tissue may be, but is not necessarily limited to, a lesion.

In some aspects, a tissue traction system configured to apply traction to target tissue at a target site includes two or more tissue-engagement elements with respect to which one or more elongate elements are operably associated to apply traction to target tissue engaged by at least one of the tissue-engagement elements. The tissue-engagement element may be in the form of a clip, clamp, or other device with structures movable (such as, without limitation, pivotable) with respect to each other to grasp tissue therebetween with or without penetrating the tissue (e.g., a device with two or more jaws movable between a tissue-receiving configuration in which the jaws are spaced apart to receive tissue therebetween, and a tissue-grasping configuration in which the jaws are moved together to grasp tissue therebetween); and/or a tissue-penetrating device such as a device with tissue-penetrating helix, barbs, hooks, tines, etc., capable of penetrating tissue to retain the tissue-engagement element with respect to tissue; and/or other configurations known to those of ordinary skill in the art, the present disclosure not being limited in this regard. It will be appreciated that terms such as engage (and other grammatical forms thereof) may be used interchangeably herein with terms such as, without limitation, couple, grasp, hold, clasp, clip, clamp, anchor, attach, affix, secure, etc. (and other grammatical forms thereof), without intent to limit. The tissue-engagement element is configured to be securely engaged with tissue to transmit to the tissue traction applied by the elongate element. In some aspects, an additional tissue-engagement element is provided to operably associate the elongate element with another location within the patient’s body to modify at least the direction of the traction force vector applied by the elongate element to the tissue at the treatment site (e.g., the target tissue). It will be appreciated that reference herein to engagement of a tissue-engagement element with tissue is intended as secure, anchoring engagement which resists withdrawal therefrom, even upon application of traction force thereto. In some aspects, the tissue-engagement elements may be considered tissue anchors.

In some aspects, the elongate element is fixedly coupled to at least one of the tissue-engagement elements to apply traction to target tissue engaged by the at least one of the tissue-engagement elements (which may be referenced as an anchor tissue-engagement element). In some aspects, one or more additional tissue-engagement elements are positioned relative to the anchor tissue-engagement element to apply traction to the target tissue at a desired directional vector, such as to lift the tissue. The elongate element may be movably (e.g., translatably) associated with the one or more additional tissue-engagement elements. In some aspects, one or more elongate elements are fixedly coupled to the anchor tissue-engagement element so that a traction force applied to the elongate element may be applied, via the elongate element, to the anchor tissue-engagement element. In some aspects, each elongate element is movably operably associated with a traction-adjusting tissue-engagement element positioned at a vector-changing location spaced apart from the target tissue. As such, movement of the elongate element while operably coupled with an associated traction-adjusting tissue-engagement element applies a directional traction force vector determined by the vector-changing location at which the associated traction-adjusting tissue-engagement element is engaged.

In accordance with various principles of the present disclosure, a tissue traction system as described herein is delivered with or for use with a medical delivery device configured for delivery of medical instruments, tools, devices, accessories, for performing a procedure with respect to the target tissue. In some aspects, a tissue traction system as disclosed herein is delivered with a tubular elongate member separate from the medical delivery device. For instance, the tubular elongate member may define a delivery lumen auxiliary to a delivery lumen and/or working channel of the medical delivery device. In some aspects, the medical delivery device is a medical scope, and the tubular elongate member is an over-the-scope (OTS) sheath configured for delivery alongside (e.g., along the exterior of) the medical scope. In some aspects, the tubular elongate member is assembled to the medical scope prior to insertion of the tubular elongate member and medical scope into the patient. As may be appreciated, delivery of the tissue traction system with a tubular elongate member which is separate from a medical delivery device allows separate and independent movement and control of the tissue traction system without being affected by movement or operation of the medical delivery device.

In some aspects, the tissue traction system is configured for delivery within a patient, such as transluminally, transcatheterally, endoscopically, etc. It will be appreciated that reference to a body lumen or passage includes naturally existing passages (e.g., the colon) as well as medically-created passages (e.g., a passage created with the use of a medical instrument, and not existing without medical intervention) or otherwise.

Various embodiments of devices, systems, and methods for applying traction to tissue will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers greater than 100 are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

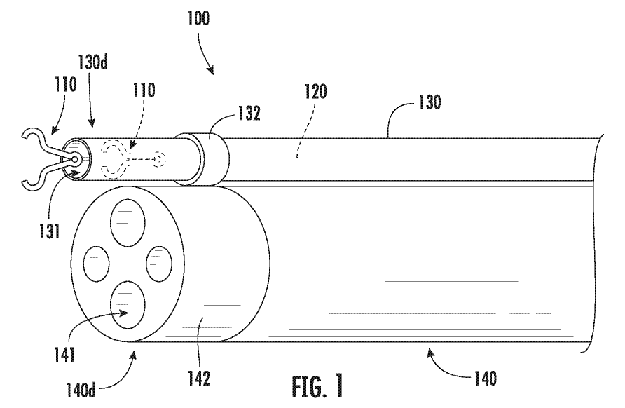

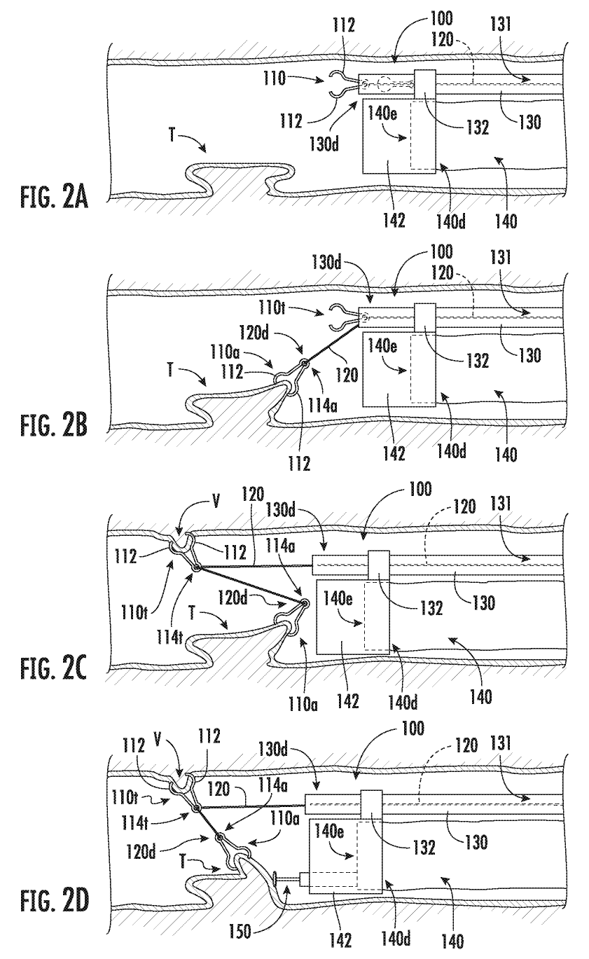

Turning now to the drawings, FIG. 1 illustrates an example of an embodiment of a tissue traction system 100 formed in accordance with various principles of the present disclosure. The illustrated tissue traction system 100 includes one or more tissue-engagement elements 110 and an elongate element 120 operatively associated with the tissue-engagement elements 110. In the example of an embodiment of a tissue traction system 100 illustrated in FIG. 1, FIGS. 2A-2D, FIGS. 3A-3B, and FIG. 4, the tissue-engagement elements 110 are in the form of a tissue engagement element with two or more jaws 112 configured to be movable away from each other into a tissue-receiving configuration to receive tissue therebetween, and towards each other to a tissue-grasping configuration to grasp tissue therebetween. However, it will be appreciated that other configurations of tissue-engagement elements 110, such as helical elements (e.g., helical tissue anchors), barbs, tines, hooks, etc., are within the scope of the present disclosure, the present disclosure not being limited by the specific configuration of a tissue-engagement element.

In some aspects, a tissue traction system 100 formed in accordance with various principles of the present disclosure is configured to be delivered with or by a delivery device such as a tubular elongate member 130, and/or medical delivery device 140 such as illustrated in FIG. 1, and discussed in further detail below. In some aspects, the tissue traction system 100 is delivered with (through or alongside) a medical delivery device through which other medical tools, instruments, devices, accessories, etc. (such terms being usable interchangeably herein without intent to limit) may be delivered to the treatment site to perform a procedure, such as illustrated in FIGS. 2A-2D. For instance, a tissue traction system 100 formed in accordance with various principles of the present disclosure may be delivered through or alongside a medical delivery device and used to apply traction to tissue with respect to which one or more instruments delivered by the medical delivery device are to be used in performing a procedure with respect to the tissue. For the sake of convenience, and without intent to limit, the tissue to which traction is to be applied is referenced herein as target tissue T along a target site. In some aspects, the target tissue T is tissue to be excised. The target tissue T may be already elevated relative to surrounding tissue (naturally projecting away from surrounding tissue), and/or a medium (e.g., a fluid such as water, saline, gel, etc.) may be injected into the target tissue T to elevate/lift the tissue (to create a “bleb”, typically before delivery of a cutting device to the target tissue T) which may facilitate creation of an initial incision to facilitate further cutting of the tissue. The target tissue T may be a lesion, but the present disclosure should not be limited in this regard. As discussed above, in some procedures, such as ESD, it is helpful to apply traction to tissue during the procedure, such as during cutting of tissue away from surrounding tissue. In some aspects, the target tissue T is separated from surrounding tissue and forms a tissue flap which is to be lifted by a tissue traction system 100 formed in accordance with various principles of the present disclosure.

In some aspects, the tissue-engagement elements 110 of a tissue traction system 100 of the present disclosure are delivered within a patient’s body transluminally, transcatheterally, endoscopically, etc., such as in a minimally-invasive surgical procedure, such as illustrated in FIG. 2A. In some aspects, the tissue traction system 100 is used to apply traction to tissue within a body lumen, such as illustrated in FIGS. 2A-2D. However, it will be appreciated that principles of the present disclosure need not be limited in this regard.

In accordance with various principles of the present disclosure, the tissue-engagement elements 110 of the tissue traction system 100 are configured to be engaged with respect to target tissue T of a patient, and the elongate element 120 is operably associated with respect to the tissue-engagement elements 110 to allow a medical professional to adjust traction force applied to the target tissue T via at least one of the tissue-engagement elements 110. In some aspects, the elongate element 120 is fixedly, non-translatably attached to at least one of the tissue-engagement elements 110 of the tissue traction system 100, and movably (e.g., translatably) operably associated with another of the tissue-engagement elements 110 of the traction system 100. For the sake of convenience, the tissue-engagement element 110 to which the elongate element 120 is non-translatably attached is referenced herein as the anchor tissue-engagement element 110a. The anchor tissue-engagement element 110a may be, though is not necessarily, the distalmost of the tissue-engagement elements 110 delivered to the target site T. It will be appreciated that reference herein to engagement of a tissue-engagement element with tissue is intended as secure, anchoring engagement which resists withdrawal therefrom, even upon application of traction force thereto.

As illustrated in FIG. 2B, the anchor tissue-engagement element 110a is engaged with target tissue T to which traction is to be applied. For instance, the jaws 112 of the anchor tissue-engagement element 110a may be shifted to a tissue-receiving configuration to receive a portion of the target tissue T therebetween, and then shifted to a tissue-grasping configuration to be engaged with and grasping the target tissue T such as illustrated in FIG. 2B. In some aspects, the anchor tissue-engagement element 110a is deployed from the tubular elongate member 130 or other delivery device, such as from the medical delivery device 140, to be separated therefrom (e.g., spaced apart from and no longer connected to, movable by, or otherwise affected by movements of the tubular elongate member 130 and/or the medical delivery device 140). In some aspects, the elongate element 120 is fixedly attached to an anchor point 114a along the anchor tissue-engagement element 110a. In the illustrated example of an embodiment, the anchor point 114a is located at an end of the anchor tissue-engagement element 110a opposite the end which engages the target tissue T (e.g., opposite the end from which jaws 112 or other feature which engages, e.g., directly engages, tissue extends). However, other locations, arrangements, configurations, etc., are within the scope of the present disclosure, the present disclosure not being limited to the illustrated examples. The anchor point 114a may include a loop, aperture, post, collar, or other structure known to those of ordinary skill in the art by which an elongate element 120 may be fixed to the anchor tissue-engagement element 110a and/or a fixation point such as a weld, bond, adhesive connection, crimp, etc., fixedly securing the elongate element 120 and the anchor tissue-engagement element 110a. In some aspects, the elongate element 120 is fixedly coupled or attached to the anchor tissue-engagement element 110a so that forces applied to the elongate element 120 are transmitted to the anchor tissue-engagement element 110a and to the target tissue T to which the anchor tissue-engagement element 110a is engaged. Typically, the elongate element 120 is fixedly coupled or attached to the anchor point 114a so that the distal end 120d of the elongate element 120 does not move with respect to the anchor tissue-engagement element 110a. As such, movement of the elongate element 120 affects the anchor tissue-engagement element 110a so that traction may be applied by the elongate element 120 to the target tissue T engaged by the anchor tissue-engagement element 110a via the anchor tissue-engagement element 110a.

In accordance with various principles of the present disclosure, the elongate element 120 is operably associated with respect to one or more additional tissue-engagement elements 110 of the tissue traction system 100 to adjust a traction force (e.g., the magnitude and/or direction of the traction force vector) on the anchor tissue-engagement element 110a and thus on the target tissue T with which the anchor tissue-engagement element 110a is engaged. In some aspects, the elongate element 120 is movably operably associated, such as translatably operably associated, with the one or more additional tissue-engagement elements 110. As such, the additional tissue-engagement elements 110 may influence the movement of the elongate element 120 without impeding the movement of the elongate element 120. For instance, the additional tissue-engagement elements 110 may affect the direction of movement (e.g., the direction of the traction force vector applied to the anchor tissue-engagement element 110a by the elongate element 120), without preventing movement of the elongate element 120, as discussed in further detail below. For the sake of convenience, and without intent to limit, the additional tissue-engagement elements 110 spaced apart from the treatment site T may be referenced herein as traction-adjusting tissue-engagement elements 110t. It will be appreciated that additional tissue-engagement elements which are engaged with tissue spaced apart from the treatment site T yet which are operably associated with an anchor tissue-engagement element 110a (engaged with the target tissue T) to apply traction to the anchor tissue-engagement element 110a may also be referenced as traction-adjusting tissue-engagement elements.

Accordingly, once the anchor tissue-engagement element 110a is attached (engaged, secured, clipped, anchored, etc.) to traction tissue T (as illustrated in FIG. 2B), an additional one of the tissue-engagement elements 110 of the tissue traction system 100 may be engaged with tissue spaced apart from the target tissue T, such as illustrated in FIG. 2C. For instance, the additional tissue-engagement element 110 may be engaged with tissue at a vector-changing location V selected so that the elongate element 120 may apply traction to the target tissue T by extending from the anchor tissue-engagement element 110a to the traction-adjusting tissue-engagement element 110t at the vector-changing location V. For instance, as used in a body lumen (as illustrated schematically in FIGS. 2A-2D), the vector-changing location V may be a portion of a tissue wall (e.g., mucosal wall) opposite (e.g., diametrically opposed to) the target tissue T. In some aspects, the elongate element 120 is movably, such as translatably, operably associated with the additional tissue-engagement element 120 along a translation point 114t along the tissue-engagement element 110. In some aspects, the translation point 114t is located at an end of the anchor tissue-engagement element 110a opposite the end which engages the target tissue T (e.g., opposite the end from which jaws 112 or other feature which engages, e.g., directly engages, tissue extends). The translation point 114t may include a loop, aperture, through hole, eyelet, and/or other structure known to those of ordinary skill in the art by which an elongate element 120 may be captured with respect to the tissue-engagement element 100, so as to remain operably associated therewith, yet also may move (e.g., translate and/or have additional freedom of movement) with respect to the tissue-engagement element 110. As illustrated in FIG. 2D, the elongate element 120 may be pulled proximally to translate an increased traction force vector on the elongate element 120 to the anchor tissue-engagement element 110a and thereby to the target tissue T. As also illustrated in FIG. 2D, the vector-changing location V may determine the direction of the traction force vector applied to the anchor tissue-engagement element 110a to apply traction to the target tissue T in a desired or medically indicate direction, such as to lift target tissue T as it is cut by a cutting device 150. The translation point 114t of the additional tissue-engagement element 110b may operate similar to a pulley, and may effect a change in the direction of the traction force vector applied to the anchor tissue-engagement element 110a and/or may affect the magnitude of the traction force vector applied to the target tissue T. It will be appreciated that although FIGS. 2A-2D illustrate deployment of the anchor tissue-engagement element 110a prior to deployment of a traction-adjusting tissue-engagement element 110t, other sequences of deployment are within the scope of the present disclosure, the present disclosure not being limited in this regard.

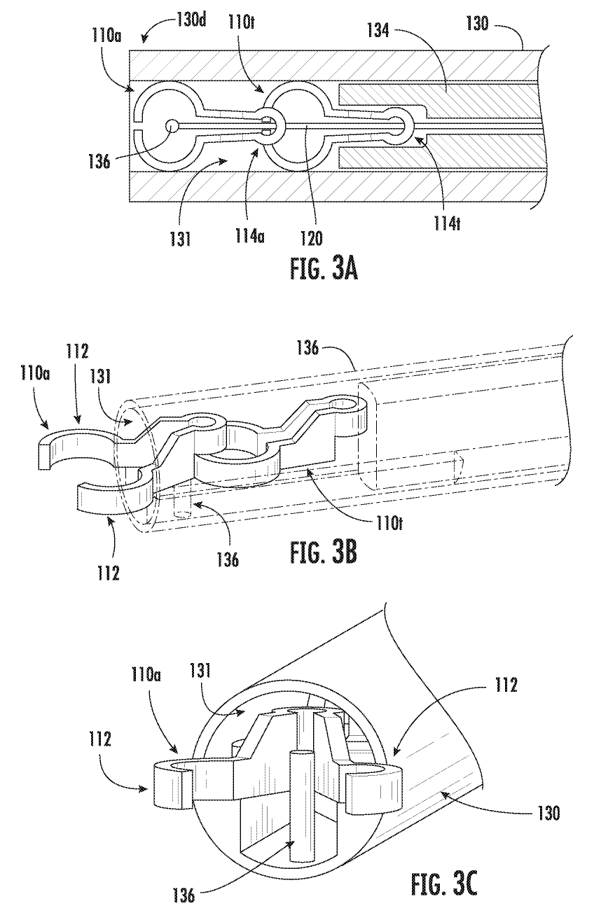

As noted above, in some aspects, the tissue traction system 100 is configured to be delivered with or by a tubular elongate member 130. In some aspects, the tubular elongate member 130 is considered to be a part or component of the tissue traction system 100. In some aspects, the tubular elongate member 130 is sufficiently flexible to be navigated into and within a patient (e.g., through curved and/or tortuous body passages within the patient) to a treatment site. The example of an embodiment of a tubular elongate member 130 illustrated in FIG. 1, FIGS. 2A-2D, and FIGS. 3A-3C defines a lumen 131 therethrough through which the tissue-engagement elements 110 are delivered. In some aspects, the tubular elongate member 130 may be in the form of a guiding channel that does not fully enclose (e.g., does not extend fully circumferentially around) the tissue-engagement elements 110. An open slot may extend longitudinally along such tubular elongate member 130 to allow free movement of the elongate element 120. In some aspects, the user may grasp and/or load or otherwise operably couple the elongate element 120 with respect to the tissue-engagement element 110 outside the patient’s body, and then distally advance the tubular elongate member 130 into the patient’s body with the elongate element 120 extending outside the tubular elongate member 130. Such configuration may allow for multiple subsequent traditional tissue-engagement element placements while engaging with the elongate element 120 while not limiting the elongate element 120 to be positioned within the tubular elongate member 130.

In some aspects, such as also illustrated in FIG. 1, the tubular elongate member 130 may be delivered along with (e.g., alongside, such as along the exterior of) an additional delivery device, such as a medical delivery device 140 configured to deliver other tools, instruments, accessories, devices, etc., to a target/deployment site within the patient. The medical delivery device 140 may be any suitable device known to those of ordinary skill in the art for delivering a medical instrument. In FIG. 1, the delivery device 140 is illustrated as an endoscope, but may be any other device, such as a tubular elongate member such as a catheter, overtube, sheath, etc. In some aspects, the tubular elongate member 130 is operably associated (e.g., slidably and/or rotatably operably coupled) with the medical delivery device 140 with a connector/coupler 132 such as a band, collar, mount, bracket, loop, band, tape, etc., which fixedly or releasably couples at least a distal end 130d of the tubular elongate member 130 with respect to a distal end 140d of the medical delivery device 140. In some aspects, the medical delivery device 140 includes an endcap 142, such as known in the art, and the tubular elongate member 130 is operably coupled with the endcap 142. As may be appreciated with reference to FIGS. 2A-2D, the endcap 142 may project distally from the distal end 140d of the medical delivery device 140 so that the end face 140e of the medical delivery device 140 may be recessed within the endcap 142 in a manner known to those of ordinary skill in the art. In some aspects, an endcap 142 may allow the tubular elongate member 130 to extend distal to the distal end 140d of the medical device 140 to better position the tissue-engagement elements 110 in the field of view of the medical device 140 while maintaining a controllable, substantially rigid construct for the tubular elongate member 130. In some aspects, the medical delivery device 140 is sufficiently flexible to be navigated into and within a patient (e.g., through curved and/or tortuous body passages within the patient) to a treatment site. In some aspects, the medical delivery device 140 is considered to be a part or component of the tissue traction system 100.

In some aspects, the tubular elongate member 130 is delivered along with (e.g., alongside, such as along the exterior of) a medical delivery device 140 defining a lumen 141 through which other medical tools, instruments, devices, accessories, etc. (such terms being usable interchangeably herein without intent to limit) may be delivered to the treatment site to perform a procedure. For instance, in some aspects, a cutting device 150 such as illustrated in FIG. 2D may be extended through a working channel 141 such as illustrated in FIG. 1. In some aspects, the tubular elongate member 130 is operably associated with the delivery device 140 such as for delivery therewith into a patient. As may be appreciated, given the limited space for transluminal navigation of delivery devices within a patient, a typical delivery device has a single working channel, if any. The tubular elongate member 130 of the tissue traction system 100 thus provides a lumen for the tissue traction system 100, in addition to the lumen 141 provided by the medical delivery device 140, for delivery of a tissue traction system 100 along with other devices or systems to be used in performing a procedure with respect to a target site within the patient. As such, the tubular elongate member 130 may be considered to provide an auxiliary lumen to the tissue traction system 100 for delivery of tissue-engagement elements 110 before, with or after, delivery of instruments through the working channel 141 of the medical delivery device 140.

In the example of an embodiment of a tissue traction system 100 illustrated in FIG. 1, FIGS. 2A-2D, and FIGS. 3A-3C, the tissue-engagement elements 110 of the tissue traction system 100 are delivered in series from the tubular elongate member 130. For instance, in some aspects, the tissue-engagement elements 110 are provided generally in a line (though not necessarily a straight line) within the lumen 131 of the tubular elongate member 130, one in front of (distal to)/behind (proximal to) an other. It will be appreciated that the anchor tissue-engagement element 110a may, but need not, be the distalmost tissue-engagement element 110 within the tubular elongate member 130 and may, but need not, be the first tissue-engagement element 110 deployed from the tubular elongate member 130. In some aspects, a pushrod 134, such as illustrated in FIG. 3A and FIG. 3B, may be disposed within the lumen 131 defined through the tubular elongate member 130. The pushrod 134 may be longitudinally translatable within the lumen 131 of the tubular elongate member 130 to engage and distally advance (e.g., distally push) the tissue-engagement elements 110 out of the tubular elongate member 130.

As noted above, in some aspects, the tissue-engagement elements 110 has two or more jaws 112 actuatable between a tissue-receiving configuration and a tissue-grasping configuration. In some aspects, the jaws 112 may be biased into the tissue-grasping configuration such that the jaws 112 remain engaged with tissue once deployed. In some aspects, the tissue-engagement elements 110 are delivered in a generally closed (partially or fully closed) configuration and are shifted into a tissue-receiving configuration when pushed (e.g., by the pushrod 134) out of the lumen 131 of the tubular elongate member 130. In some aspects, the tubular elongate member 130 and/or the pushrod 134 may be configured to actuate the jaws 112 of a tissue-engagement element 110 into a tissue-receiving configuration to receive tissue to be grasped between the jaws 112 as the tissue-engagement element 110 is being pushed out of the lumen 131 of the tubular elongate member 130. For instance, as illustrated in FIG. 3A, FIG. 3B, and FIG. 3C, an actuation post 136 may extend radially inwardly from a wall defining the tubular elongate member 130. The actuation post 136 extends a sufficient distance radially-inwardly to engage the jaws 112 of the distalmost tissue-engagement element 110 as the distalmost tissue-engagement element 110 is advanced distally from the tubular elongate member 130 to shift the jaws 112 into a tissue-receiving configuration, as illustrated in FIG. 3B and in the detail view in FIG. 3C. In some aspects, the anchor point 114a of the anchor tissue-engagement element 110a and/or the translation point 114t of the traction-adjusting tissue-engagement element 110t is configured and/or positioned to not be engaged by the actuation post 136 (e.g., radially spaced closer to the center of the lumen 131 of the tubular elongate member 130 so as to be movable with respect to the actuation post 136) so that the tissue-engagement elements 110a, 110t may be distally advanced past the actuation post 136 for deployment. Alternatively, the jaws 112 may be arranged with respect to (e.g., crossed over) each other within the lumen 131 of the tubular elongate member 130 to be in a tissue-receiving configuration when partially extended from the distal end 130d of the tubular elongate member 130. The jaws 112 may be held in the tissue-receiving configuration against a biasing force which biases the jaws 112 into a tissue-engaging configuration. Once deployed out of the lumen 131 of the tubular elongate member 130, the biasing force shifts the jaws 112 into a tissue-engaging configuration for deployment of the tissue-engagement element 110 secured or anchored or otherwise engaged with respect to tissue. An example of a configuration of a tubular elongate member 130 and a configuration of a tissue-engagement element 110 in which the tissue-engagement element 110 is actuated into a tissue-receiving configuration upon delivery, and may then be deployed in a tissue-engaging configuration secured with respect to tissue is shown and described in U.S. Patent 10,973,506 to Smith et al., issued on Apr. 13, 2021, and titled Systems, Devices, And Related Methods For Retracting Tissue, the entire disclosure of which is hereby incorporated herein by reference in its entirety for all purposes.

Once the tissue-engagement element 110 has been deployed out of the tubular elongate member 130 (e.g., fully out of the tubular elongate member 130, such as fully out of the lumen 131 of the tubular elongate member 130), the jaws 112 may return to a tissue-grasping configuration (e.g., biased towards a closed configuration) engaging tissue. In some aspects, the tissue-engagement elements 110 are deployed in a tissue-receiving configuration (e.g., with the jaws 112 spaced apart to receive tissue therebetween), and a retainer is placed over the jaws 112 or the jaws 112 are otherwise reconfigured (e.g., crimped, deformed, etc.), such as in any manner known to those of ordinary skill in the art, once engaged with tissue (e.g., once tissue is positioned between the jaws 112) to be retained with respect to (e.g., to remain engaged with) the tissue once deployed.

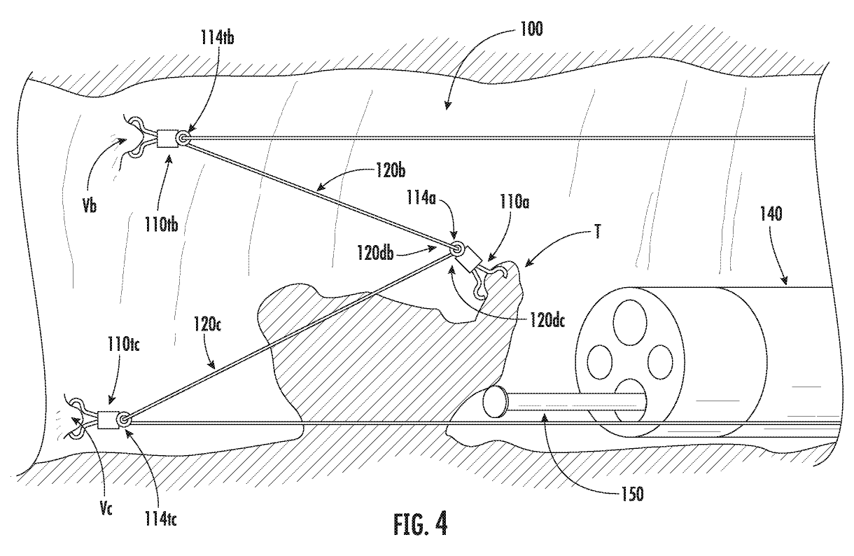

As may be appreciated, in accordance with various principles of the present disclosure, further tissue-engagement elements 110 may be provided in a tissue traction system 100 of the present disclosure than an anchor tissue-engagement element 110a and only one additional tissue-engagement element 110 (such as illustrated in FIGS. 2A-2D). For instance, in the example of an embodiment illustrated in FIG. 4, an anchor tissue-engagement element 110a and two additional tissue-engagement elements 110 are deployed by a tissue traction system 100 formed in accordance with various principles of the present disclosure. It will be appreciated that the tissue traction system 100 illustrated in FIG. 4 is illustrated already deployed with respect to tissue, and may be delivered and/or deployed in a manner such as described above with respect to FIGS. 2A-2D, reference being made thereto for the sake of brevity and without intent to limit, or in any other desired manner known to those of ordinary skill in the art. In some aspects, the additional tissue-engagement elements 110 are deployed sequentially from a lumen 131 defined in a tubular elongate member 130, such as illustrated in FIGS. 3A-3C.

In some aspects, each of the additional tissue-engagement elements 110 illustrated in FIG. 4 has a separate elongate element 120 each fixedly attached to the anchor tissue-engagement element 110a. For instance, as illustrated in FIG. 4, each additional traction-adjusting tissue-engagement element 110tb, 110tc, etc. (additional traction-adjusting tissue-engagement elements 110 which are not illustrated yet which may be deployed in a manner as appreciated by those of ordinary skill in the art), includes an associated elongate element 120b, 120c, etc. More particularly, a traction-adjusting tissue-engagement element 110tb may be engaged with tissue at a vector-changing location Vb spaced apart from the target tissue T, with the distal end 120db of an elongate element 120b fixedly coupled with an anchor tissue-engagement element 110a engaged with target tissue T and operably associated with the traction-adjusting tissue-engagement element 110tb. An additional traction-adjusting tissue-engagement element 110tc may be engaged with tissue at a vector-changing location Vc spaced apart from the target tissue T as well as from the vector-changing location Vb, with a distal end 120dc of an elongate element 120c (separate and distinct from the elongate element 120b operably associated with the traction-adjusting tissue-engagement element 120b) fixedly coupled with the anchor tissue-engagement element 110a and operably associated with the additional traction-adjusting tissue-engagement element 110tc. Similar to the example of an embodiment illustrated in FIGS. 2A-2D, the elongate element 120b may be translatably operably associated with the traction-adjusting tissue-engagement element 110tb via a translation point 114tb to modify the traction force vector applied to the elongate element 120b and the anchor tissue-engagement element 110a, and/or the elongate element 120c may be translatably operably associated with the additional traction-adjusting tissue-engagement element 110tc via a translation point 114tc to modify the traction force vector applied to the elongate element 120c and the anchor tissue-engagement element 110a. In some aspects, entanglement of elongate elements 120 associated with the various tissue-engagement elements 110 may be minimized or prevented by deploying a traction-adjusting tissue-engagement element 110tb, 110tc before deploying the anchor tissue-engagement element 110a and then deploying the next traction-adjusting tissue-engagement element 110tc, 110tb after deploying the anchor tissue-engagement element 110a. The elongate element 120 slides with respect to the traction-adjusting tissue-engagement elements 110tb and 110tc yet is fixed with respect to the anchor tissue-engagement element 110a. Deployment in such order may allow placement of the traction-adjusting tissue-engagement elements 110tb, 110tc laterally across the anatomy in the area of the treatment site without deployment of an additional traction-adjusting tissue-engagement element 110tc, 110tb crossing over a previously-placed traction-adjusting tissue-engagement element 110tb, 100tc (which may cause tangling of the elongate element 120).

As may be appreciated, the size of the flap of tissue formed by cutting the target tissue T with the cutting device 150 increases as the cutting device 150 continues to cut the target tissue T, typically requiring the magnitude and/or direction of the traction force vector on the target tissue T to be adjusted to keep the target tissue T from interfering with operation of the cutting device 150. The deployment of more than one additional tissue-engagement element 110 allows the traction force on the target tissue T to be adjusted/modified during the course of a procedure. Each additional tissue-engagement element 110 may be engaged with a different vector-changing location V to adjust the direction of the traction force vector applied to the target tissue T. In some aspects, the tissue-engagement elements 110 (e.g., the anchor tissue-engagement element 110a and the traction-adjusting tissue-engagement elements 110t) are delivered to the target tissue T by a tubular elongate member 130 separate from (e.g., auxiliary to) a medical delivery device 140 with which medical instruments for performing a procedure with respect to the target tissue T are delivered.

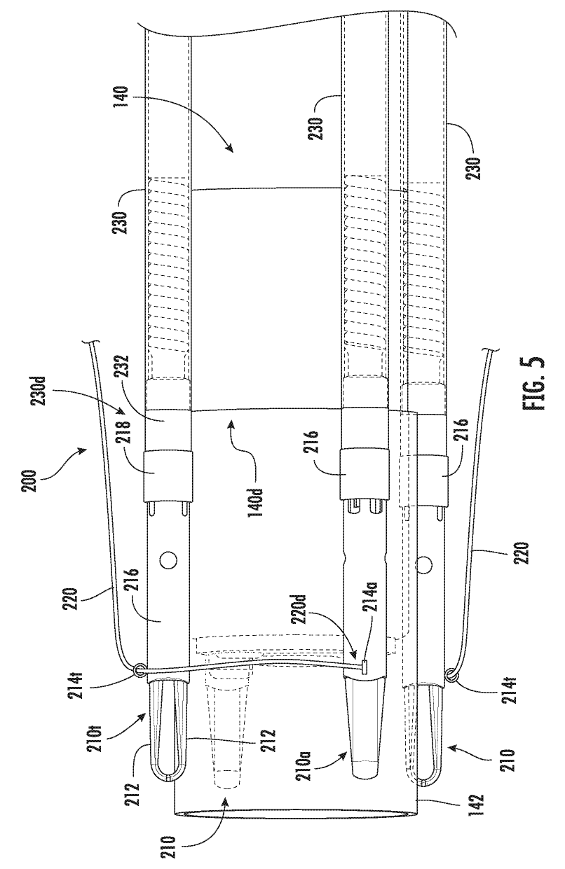

In some aspects, instead of more than one traction-adjusting tissue-engagement element 110t being delivered in series with an anchor tissue-engagement element 110a within a lumen 131 of a tubular elongate member 130, each tissue-engagement element 220 of a tissue traction system 200 formed in accordance with various principles of the present disclosure may be independently delivered, such as by a dedicated tubular elongate member 230 (e.g., which may deliver only one tissue-engagement element 210), as illustrated in FIG. 5. In some aspects, the tissue-engagement elements 210 are delivered spaced apart around a medical delivery device (e.g., not in a line or in series), as illustrated in FIG. 5. In some aspects, an anchor tissue-engagement element 210a and one or more traction-adjusting tissue-engagement elements 210t are arranged circumferentially (optionally spaced apart from one another) around a medical delivery device 140. In some aspects, at least one of the tissue-engagement elements 210 is delivered by a tubular elongate member 230 extending along the exterior of the medical delivery device 140. In some aspects, each tissue-engagement element 210 is delivered by a tubular elongate member 230 extending along the exterior of the medical delivery device 140, such as with a one-to-one correspondence of tissue-engagement element 210 and tubular elongate member 230. For instance, a pair of tubular elongate members 230 may be provided for each pair of an anchor tissue-engagement element 110a and a traction-adjusting tissue-engagement element 110t. More particularly, as illustrated in FIGS. 5-7, an anchor tissue-engagement element 210a, with an anchor point 214a to which a distal end 220d of an elongate element 220 may be fixedly coupled or attached, may be delivered in a tubular elongate member 230 separate from and independent of (e.g., adjacent, such as spaced apart from) another tubular elongate member 230 which delivers a traction-adjusting tissue-engagement element 210t with a translation point 214t allowing movable operable association of the elongate element 220 therewith (such as described above with respect to the traction-adjusting tissue-engagement element 110t and translation point 114t and elongate element 120, reference being made to the description thereof, for the sake of brevity and without intent to limit, as applicable mutatis mutandis to the traction-adjusting tissue-engagement element 210t and translation point 214t and elongate element 220). In some aspects, the anchor point 214a and/or the translation point 214t is located along an exterior of the associated tissue-engagement element 210 (anchor tissue-engagement element 210a or traction-adjusting tissue-engagement element 210t), such as along the longitudinal extent of the tissue-engagement element 210 between the ends (e.g., terminal ends) thereof. In some aspects, the anchor point 214a and/or the translation point 214t extends substantially radially outwardly from the associated tissue-engagement element 210. In some aspects, the anchor point 214a and/or the translation point 214t is operably associated with (e.g., mounted on, extends from, etc.) the capsule 216 of the associated tissue-engagement element 210.

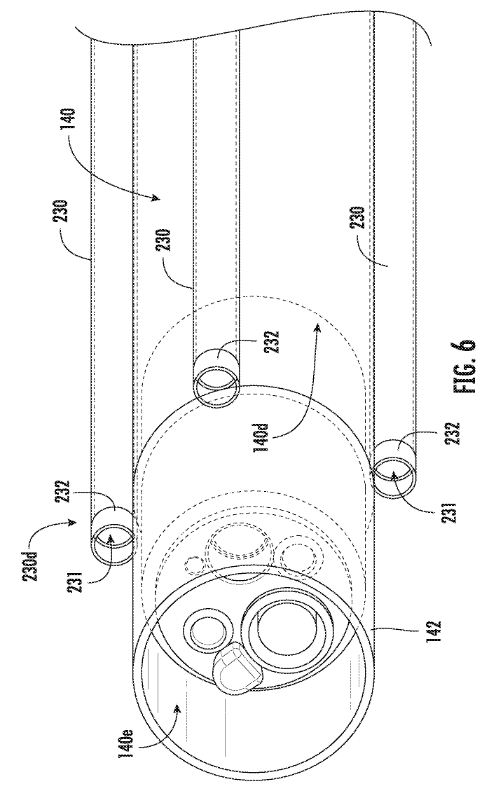

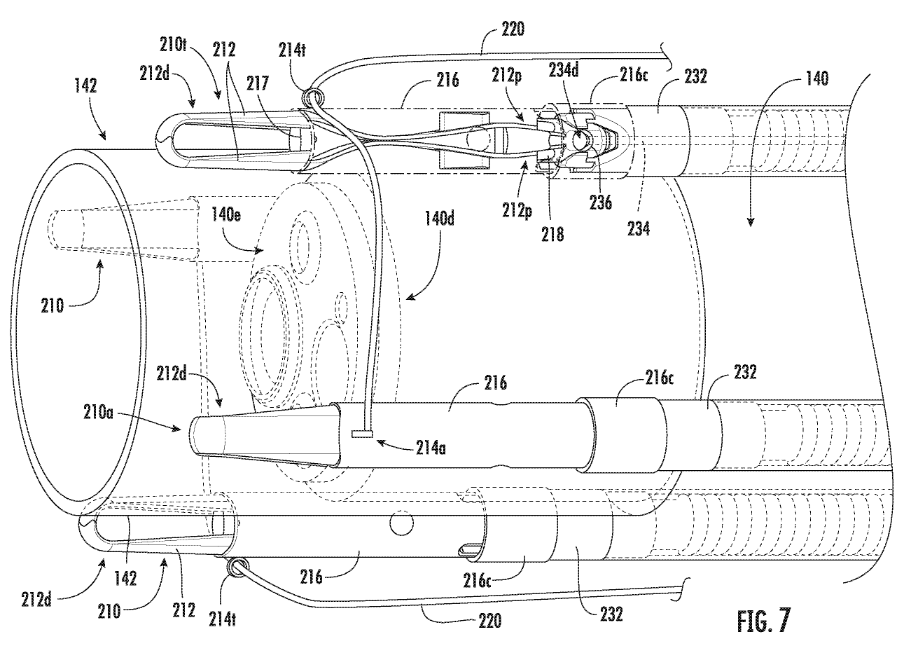

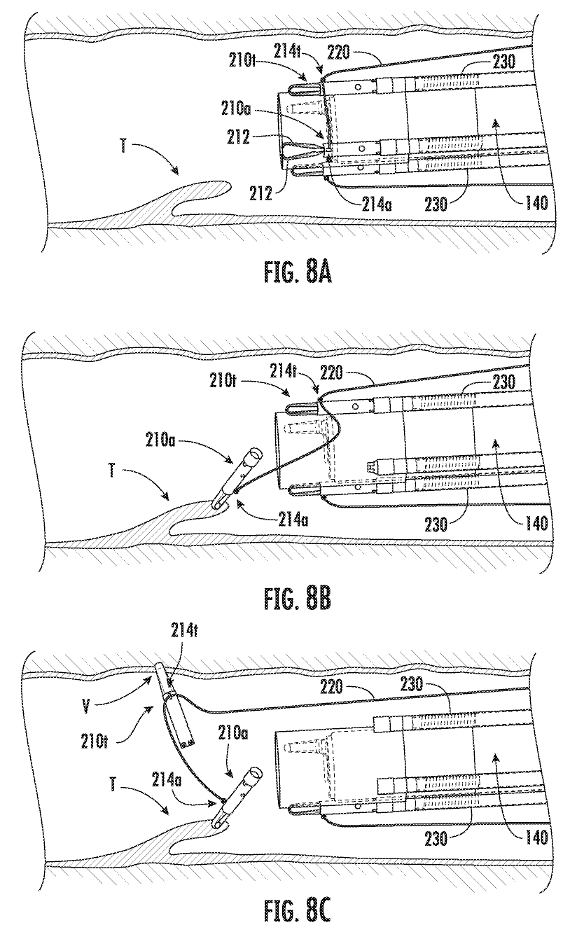

As illustrated in FIG. 5, FIG. 6, FIG. 7, and FIGS. 8A-8C, the tubular elongate members 230 which deliver the tissue-engagement elements 210 of the illustrated example of an embodiment of a tissue traction system 200 may be positioned circumferentially about a medical delivery device 140, optionally, though not necessarily, evenly spaced apart from one another. In some aspects, one or more of the tubular elongate members 230 is operably associated with (slidably and/or rotatably coupled with) the medical delivery device 140 with a connector/coupler 232 such as a band, collar, mount, bracket, loop, band, etc., which fixedly or releasably couples at least a distal end 230d of the tubular elongate member 230 with respect to a distal end 140d of the medical delivery device 140. In some aspects, the medical delivery device 140 includes an endcap 142, such as described above, and the tubular elongate members 230 are operably coupled with the endcap 142. As may be appreciated with reference to FIG. 6, the end face 140e of the medical delivery device 140 may be recessed within the endcap 142 in a manner known to those of ordinary skill in the art. In some aspects, once the target tissue T has been completely removed/excised from surrounding tissue, the endcap 142 may be used to capture the separated target tissue T for withdrawal from the patient’s body (e.g., for biopsy).

As may further be appreciated with reference to FIG. 6, each of the tubular elongate members 230 defines a lumen 231 therethrough for accommodating a portion of a tissue-engagement element 210 (e.g., a tissue-grasping portion and/or an actuating portion). For instance, each of the tissue-engagement elements 210 illustrated in FIG. 7 has a pair of jaws 212 axially translatable with respect to a capsule 216 (e.g., axially translatable within a lumen 217 defined in the capsule 216). The illustrated examples of embodiments of tissue-engagement elements 210 are configured to be actuatable (such as by a medical professional accessing a component of the tissue-engagement element 210 or a component operably coupled with the tissue-engagement element 210) to shift the configuration of the jaws 212 of the tissue-engagement element 210. In some aspects, an actuator 234 is operably coupled with respect to the proximal ends 212p of the jaws 212 to pull (or at least maintain) the proximal ends 212p of the jaws 212 within the capsule 216 in a tissue-grasping configuration (e.g., closer together to grasp tissue therebetween, such as a closed configuration), or to push the distal end 212d of the jaws 212 distally out of the capsule 216 a sufficient distance to allow the jaws 212 to move to a tissue-receiving, spaced apart (e.g., open) configuration, such as illustrated in FIG. 8A. In some aspects, a collar 216c is provided along the capsule 216 to prevent the tissue-engagement element 210, and, particularly the capsule 216, from moving proximally into the lumen 231 of the tubular elongate member 230 during actuation of the jaws 212. In some aspects, the actuator 234 is releasable from the jaws 212 to deploy the tissue-engagement element 210 separate and apart from the actuator 234. For instance, in some aspects, the actuator 234 has a distal end 234d operably coupled to the tissue-engagement element 210 to permit separation of the actuator 234 from the tissue-engagement element 210. In some aspects, the distal end 234d of the actuator 234 is frangible so that proximal movement of the actuator 234 with respect to the jaws 212 causes the distal end 234d to break off from the remaining proximally extending portion of the actuator 234 (extending proximally through the lumen 231 of the tubular elongate member 230, such as to be controlled/pulled from outside the patient’s body) to separate the jaws 212 from the remaining portion of the actuator 234. In some aspects, the distal end 234d of the actuator 234 is fixed within a yoke 236 which, in turn, is releasably coupled with a tension breaker 218 operably coupled with the jaws 212. In some aspects, the yoke 236 is separable from the tension breaker 218 to fix the jaws 212 in a tissue-grasping configuration, and then the yoke 236 is separable from the actuator 234 upon separation of the distal end 234d from the remaining proximal portion of the actuator 234, to separate the jaws 212, and the tissue-engagement element 210 in general, from the actuator 234. Various configurations of tissue-engagement members may be used in conjunction with the tissue traction system 100 of the present disclosure, such as any of the clips disclosed in U.S. Patent 7,494,461, issued Feb. 24, 2009, to Wells et al., and titled “THROUGH THE SCOPE TENSION MEMBER RELEASE CLIP”; U.S. Patent 8,080,021, issued Dec. 20, 2011, to Griego, and titled “MULTIPLE CLIP DEPLOYMENT MAGAZINE”; and U.S. Patent 8,162,959, issued Apr. 24, 2012, to Cohen et al., and titled “SINGLE STAGE HEMOSTASIS CLIPPING DEVICE”, all of which patents are incorporated by referenced herein in their entireties for all purposes. It will be appreciated, however, that the tissue-engagement elements 210 of the tissue traction system 200 of FIGS. 5-7 and FIGS. 8A-8C may be in any of a variety of other configurations or forms, the present disclosure not being limited in this regard.

An example of use of a tissue traction system 200 as illustrated in FIGS. 5-7 is illustrated in FIGS. 8A-8C. As illustrated in FIG. 8A, a tissue traction system 200 such as illustrated in FIGS. 5-7 may be delivered within a patient’s body transluminally, transcatheterally, endoscopically, etc., such as in a minimally invasive surgical procedure. For instance, the schematically illustrated environment in which the illustrated example of an embodiment of a tissue traction system 200 is deployed in FIGS. 8A-8C is a body lumen, similar to the schematically illustrated environment illustrated in FIGS. 2A-2D (with the exception of a slightly different configuration of target tissue T, the present disclosure not being limited in this regard). One of the anchor tissue-engagement elements 210a may be distally advanced with respect to its associated capsule 216 (typically once delivered to target tissue T, and generally not while the tissue traction system 200 is being advanced within the patient to the target tissue T) to be in a tissue-receiving configuration, as illustrated in FIG. 8A. The anchor tissue-engagement element 210a may be engaged with target tissue T (e.g., by advancing the anchor tissue-engagement element 210a distally out of the tubular elongate member 230 and/or by distally advancing the associated tubular elongate member 230 and/or by distally advancing the medical delivery device 140) and shifted into a tissue-grasping configuration, such as illustrated in FIG. 8B. In some aspects, the anchor tissue-engagement element 210a is deployed from the tubular elongate member 230 or other delivery device, such as from the medical delivery device 140, to be separated therefrom (e.g., spaced apart from and no longer connected to, movable by, or otherwise affected by movements of the tubular elongate member 230 and/or the medical delivery device 140). In some aspects, the anchor tissue-engagement element 210a may be separated from the actuator 234 used to shift the anchor tissue-engagement element 210a between the tissue-receiving configuration and the tissue-grasping configuration (such as described above). The traction-adjusting tissue-engagement element 210t may then be engaged with tissue in a similar manner as described with respect to the anchor tissue-engagement element 210a, but at a vector-changing location V spaced apart from the target tissue T, such as illustrated in FIG. 8C. Because the elongate element 220 is fixed with respect to the anchor point 214a of the anchor tissue-engagement element 210a and movable (e.g., translatable) with respect to the translation point 214t of the traction-adjusting tissue-engagement element 210t, the traction-adjusting tissue-engagement element 210 may be used to adjust the traction force vector applied by the elongate element 220 to the anchor tissue-engagement element 210a and thus to the target tissue T in a manner as described above, reference therefore being made, for the sake of brevity and without intent to limit, to the above descriptions with respect to the tissue traction system 100 as applicable mutatis mutandis to the use of the tissue traction system 200 of FIGS. 5-7 and FIGS. 8A-8C. For instance, traction force may be applied to the elongate element 220 (e.g., by applying traction force to a proximal end of the elongate element 220 positioned outside the patient’s body, or by applying a traction force to a member positioned outside the patient’s body operably coupled with the elongate element 220 to apply traction force to the elongate element 220). Another traction force vector adjusting pair of an anchor tissue-engagement element 210a and a traction-adjusting tissue-engagement element 210t of the tissue traction system 100 may be deployed to further adjust the direction or magnitude of the traction force vector applied to the target tissue T as desired and/or medically indicated. In some aspects, the elongate elements 220 of the tissue-engagement elements 210 of the example of an embodiment of a tissue traction system 200 illustrated in FIGS. 5-7 and FIGS. 8A-8C extends outside/external to the tubular elongate member 230 used to deliver the tissue-engagement elements 210. As such, movement of the elongate elements 220 may be independent of (separate and spaced apart from and not affected by) movement of the medical delivery device 140, such as during delivery and use of additional medical instruments delivered by the medical delivery device 140.

Although embodiments of the present disclosure may be described with specific reference to medical devices and systems and methods of applying traction to tissue in association with procedures for excising and lifting tissue, it should be appreciated that devices, systems, and methods described herein may be used to apply traction and/or to move tissue in conjunction with other procedures, or for other purposes. Various further benefits of the various aspects, features, components, and structures of traction systems, devices, and methods such as described above, in addition to those discussed above, may be appreciated by those of ordinary skill in the art.

It should be understood that the various embodiments illustrated in the figures have several separate and independent features, which each, at least alone, has unique benefits which are desirable for, yet not critical to, the presently disclosed traction system, devices, and methods. It will be appreciated that various modifications to the above-described tissue traction systems are within the scope of the present disclosure. For instance, more than one anchor tissue-engagement element may be engaged with target tissue, with one or more elongate element coupled thereto, each elongate element being movably operably associated with an additional traction-adjusting tissue-engagement element engaged with tissue at a vector-changing location spaced apart from the target tissue. It will be appreciated, as mentioned above, that the tissue-engagement elements may be in various other forms or configurations than illustrated and described. The various medical devices, instruments, tools, etc. of the present disclosure are not limited, and may include a variety of medical devices, instruments, tools, etc., for accessing anatomical structures within a patient’s body, including, for example, endoscopes, duodenoscopes, catheters, ureteroscopes, bronchoscopes, colonoscopes, arthroscopes, cystoscopes, hysteroscopes, and the like. Various medical devices, instruments, tools, etc. may be used in conjunction with tissue traction system, and associated devices and methods, formed in accordance with principles of the present disclosure. For instance, such instruments or tools may be used to perform a procedure or operation which is either diagnostic or therapeutic or both, such as grasping, resecting, dissecting, retracting, cutting, and/or otherwise manipulating tissue. Such instruments or tools may include, without limitation, graspers (e.g., a rotatable grasping clip, such as a RESOLUTION™ clip device sold by Boston Scientific Corporation, with a pair of jaws/arms, etc.), a cutting tool (e.g., knife, electrosurgical knife, electrocautery device, scissors), snares, etc.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and / or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader’s understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A tissue traction system comprising:
two or more tissue-engagement elements each configured to engage tissue within a patient’s body; and
at least one elongate element;
wherein:
the two or more tissue-engagement elements include at least one anchor tissue-engagement element configured to engage target tissue within a patient’s body to which traction is to be applied, and at least one traction-adjusting tissue-engagement element;
said at least one elongate element is fixedly coupled to said anchor tissue-engagement element;
at least said anchor tissue-engagement element is deployed from a delivery device to be separated from the delivery device when engaged with the target tissue; and
said elongate element is movably operably associated with said at least one traction-adjusting tissue-engagement element to adjust a traction force vector applied to said anchor tissue-engagement element by said elongate element to apply traction to the target tissue.

2. The tissue traction system of claim 1, further comprising a tubular elongate member defining a lumen therethrough, wherein said two or more tissue-engagement elements are deliverable within the lumen of said tubular elongate member to a target site within the patient’s body.

3. The tissue traction system of claim 2, wherein said tubular elongate member is configured to be operably associated with a medical delivery device configured to deliver a medical instrument to the target site.

4. The tissue traction system of claim 3, wherein said tubular elongate member is operably associated with an exterior of the medical delivery device for delivery alongside the exterior of the medical delivery device with the lumen of said tubular elongate member providing an auxiliary lumen to the medical delivery device.

5. The tissue traction system of claim 2, further comprising a push rod longitudinally translatable within the lumen of the tubular elongate member to distally advance the tissue-engagement elements out of the tubular elongate member.

6. The tissue traction system of claim 1, wherein said at least one traction-adjusting tissue-engagement element includes at least a first traction-adjusting tissue-engagement element and a second traction-adjusting tissue-engagement element, and said at least one elongate element includes a first elongate element movably coupled with said first traction-adjusting tissue-engagement element and a second elongate element movably coupled with said second traction-adjusting tissue-engagement element.

7. The tissue traction system of claim 6, wherein said first elongate element and said second elongate element are both fixedly coupled with said at least one anchor tissue-engagement element.

8. The tissue traction system of claim 6, wherein said at least one anchor tissue-engagement element, said first traction-adjusting tissue-engagement element, and said second traction-adjusting tissue-engagement element are deliverable in series within a lumen defined in a tubular elongate member.

9. The tissue traction system of claim 6, wherein:
said at least one anchor tissue-engagement element comprises a first anchor tissue-engagement element and a second anchor tissue-engagement element;
said first elongate element is fixedly coupled with said first anchor tissue-engagement element; and
said second elongate element is fixedly coupled with said second anchor tissue-engagement element.

10. The tissue traction system of claim 9, further comprising a first tubular elongate member configured for delivering said first anchor tissue-engagement element, and a second tubular elongate member configured for delivering said first traction-adjusting tissue-engagement element said second anchor tissue-engagement element, and said second anchor tissue-engagement element.

11. The tissue traction system of claim 10, wherein said plurality of tubular elongate members are operably associable about the circumference of a medical delivery device.

12. The tissue traction system of claim 1, wherein said at least one elongate element is fixedly coupled with respect to an anchor point along said anchor tissue-engagement element.

13. The tissue traction system of claim 1, wherein said at least one elongate element is slidably associated with said at least one traction-adjusting tissue-engagement element.

14. The tissue traction system of claim 13, wherein said at least one elongate element is fixedly coupled to an anchor point on said anchor tissue-engagement element to transmit a traction force to said anchor tissue-engagement element to apply traction to the target tissue, and is slidable through a translation point configured to capture said elongate element with respect to said traction-adjusting tissue-engagement element yet allow translation of said elongate element with respect to said translation point.

15. A tissue traction system comprising:
two or more tissue-engagement elements each configured to engage tissue within a patient’s body;
at least one elongate element; and
a tubular elongate member configured to deliver said two or more tissue-engagement elements and said at least one elongate element into a patient’s body;
wherein:
the two or more tissue-engagement elements include at least one anchor tissue-engagement element configured to engage target tissue within a patient’s body to which traction is to be applied, and at least one traction-adjusting tissue-engagement element;
said at least one elongate element is fixedly coupled to said anchor tissue-engagement element;
at least said anchor tissue-engagement element is deployed from a delivery device to be separated from the delivery device when engaged with the target tissue; and
said elongate element is movably operably associated with said at least one traction-adjusting tissue-engagement element to adjust a traction force vector applied to said anchor tissue-engagement element by said elongate element to apply traction to the target tissue.

16. The tissue traction system of claim 15, wherein said tubular elongate member is flexible to be navigated within curved passages within the patient’s body, and defines a lumen therethrough, wherein said two or more tissue-engagement elements are deliverable in series within the lumen of said tubular elongate member to a target site within the patient’s body.

17. The tissue traction system of claim 15, further comprising a plurality of tubular elongate members operably associable with a medical delivery device, wherein:
said at least one anchor tissue-engagement element comprises a first anchor tissue-engagement element deliverable by a first of said plurality of tubular elongate members; and
said at least one traction-adjusting tissue-engagement element comprises a first traction-adjusting tissue-engagement element deliverable by a second of said plurality of tubular elongate members.

18. The tissue traction system of claim 17, further comprising:
a second anchor tissue-engagement element deliverable by a third of said plurality of tubular elongate members; and
a second traction-adjusting tissue-engagement element deliverable by a fourth of said plurality of tubular elongate members.

19. A method of applying traction to target tissue within a patient’s body, said method comprising:
deploying an anchor tissue-engagement element at a target site within the patient’s body by separating the anchor tissue-engagement element from a first delivery device and engaging the anchor tissue-engagement element with target tissue at the target site;
deploying a traction-adjusting tissue-engagement element by separating the traction-adjusting tissue-engagement element from a delivery device, which is the same as or delivered with the first delivery device, and engaging the traction-adjusting tissue-engagement element with tissue at a vector-changing location within the patient’s body spaced apart from the target site; and
extending an elongate element, having a distal end fixedly coupled to the anchor tissue-engagement element, from the anchor tissue-engagement element to the traction-adjusting tissue-engagement element; and
moving the elongate element with respect to the traction-adjusting tissue-engagement element to adjust a traction force vector applied by the elongate element to the anchor tissue-engagement element to apply traction to the target tissue.

20. The method of claim 19, wherein the anchor tissue-engagement element and the traction-adjusting tissue-engagement element are delivered external to a medical delivery device defining a lumen through which additional medical instruments are deliverable to the target site, and the elongate element extends external to the medical delivery device, said method comprising applying a traction force to the elongate element from outside the patient’s body.
